(19) United States
(12) Patent Application Publication
(10) Pub. No.: US 2026/0315249 A1
(43) Pub. Date: Oct. 15, 2026

(54) PASSIVE MECHANISM FOR PROSTHESES AND ORTHOSES

(71) Applicant: LITTLE ROOM INNOVATIONS, Ann Arbor, MI (US)

(72) Inventors: Harrison Logan Bartlett, Atlanta, GA (US); Brian Edward Lawson, Ann Arbor, MI (US)

(21) Appl. No.: 18/834,673

(22) PCT Filed: Feb. 21, 2023
(86) PCT No.: PCT/US2023/013483

§ 371 Date: Jul. 31, 2024

Related U.S. Application Data
(60) Provisional application No. 63/313,957, filed on Feb. 25, 2022.

(51) Int. Cl. A61F 2/66; A61F 2/50; A61F 2/74

(52) U.S. Cl. CPC A61F 2/6607; A61F 2/748; A61F 2002/5043; A61F 2002/5072; A61F 2002/6614

(57) ABSTRACT

Various implementations include a leg prosthesis or orthosis device. The device includes a shank member, a foot member, a connecting link, and a sliding link. The connecting link is movably coupled to the shank member at a first location and is moveably coupled to the foot member at a second location. The sliding link is constrained to move along a sliding axis relative to the shank member and is rotatably coupled to the foot member at a third location.

### BACKGROUND

Over half of lower limb prosthesis users report falling at least once per year. Furthermore, most falls experienced by prosthesis users occur during level ground walking as a result of a trip, slip, or problem caused by the prosthetic device. The risk of falls can be mitigated through dorsiflexion of the foot during the swing phase, which promotes increased ground clearance, thereby minimizing the risk of a scuff, trip, or stumble. Multiple studies link increased ground clearance with a reduction in fall risk both in a laboratory setting and in the real world.

Multiple devices provide increased ground clearance functionality such as the passive hydraulic prosthetic ankle described in U.S. Pat. No. 7,942,935. Such a passive hydraulic ankle yields in dorsiflexion during the stance phase of gait and remains in a dorsiflexed state throughout the swing phase of gait. However, such an approach for providing swing phase dorsiflexion comes at the price of energetic inefficiency during the stance phase. Specifically, the prosthetic ankle yields during stance phase in a dissipative fashion, and the energy spent yielding the prosthesis is not returned to the prosthesis user at terminal stance. As such, passive hydraulic ankles such as the ones described in U.S. Pat. Nos. 7,942,935 and 6,443,993 reduce the risk of falls due to their swing phase dorsiflexion functionality but at the price of energetic inefficiency. This dissipative stance phase yielding is in contrast to energy storage and release prosthetic feet which utilize spring-like structures to elastically store and release energy during stance phase. As such, there exists a need for a prosthetic/orthotic ankle joint mechanism that allows for swing phase dorsiflexion without sacrificing stance phase elastic energy storage and release. Furthermore, it is advantageous for this functionality to be achieved in a compact, lightweight, and durable fashion.

Numerous approaches have been applied to the problem of swing phase dorsiflexion. For example, prosthetic devices, such as those disclosed in U.S. Pat. No. 10,299,943, U.S. Published Application Number 2005/0192677, U.S. Pat. No. 10,772,742, and U.S. Published Application Number 2021/018671, have been developed which use powered mechatronic systems to drive the ankle joint during the swing phase of gait to achieve swing phase dorsiflexion. These powered devices, however, require motors, transmissions, power electronics, and batteries to operate. As such, these devices tend to be large, heavy, and exhibit considerable audible noise. Another approach to providing swing phase dorsiflexion is to utilize a mechatronic clutching system in conjunction with a spring-like element to lock the joint during the stance phase and unlock the ankle during swing phase, during which time the ankle is biased towards a dorsiflexed position through a spring like element. Examples of devices that employ this mechatronic clutch approach include the ones described in European Patent Number 3,613,389, U.S. Pat. Nos. 10,376,388, 11,033,408, 10,709,583, and 9,549,827. These devices require the use of mechatronic elements such as a battery, power electronics, and an electromechanical actuator to operate, and as such, are typically, large, heavy, and exhibit significant audible noise.

To combat the issues presented by mechatronic systems, multiple passive devices have been developed which utilize weight-activated clutch systems to provide stance phase stiffness and swing-phase dorsiflexion. These devices include a unidirectional clutch that, when engaged, allows for plantarflexion but prevents dorsiflexion. The clutch is engaged when the user's weight is applied to the device during the stance phase and is disengaged when the user's weight is removed. When the clutch element is disengaged, the ankle joint is free to dorsiflex, and a spring element biases the ankle towards dorsiflexion. When the clutch is engaged, the ankle freely plantarflexes to conform to the ground, but is not able to dorsiflex due to the unidirectional clutch, thereby allowing for stance phase stiffness. Devices in this category, such as U.S. Pat. Nos. 8,480,760, 9,289,316, International Application Number WO 2019/028388, and U.S. Pat. No. 8,597,369, have utilized various clutch mechanisms to achieve this functionality. Devices in this category suffer from mechanical complexity and reliability concerns due to the use of small clutch mechanisms that must sustain high forces.

Other distinct approaches for providing swing phase dorsiflexion include a four-bar linkage mechanism, or prosthetic devices with articulated toe joints, such as those disclosed in U.S. Pat. Nos. 9,439,786, 10,342,680, International Application Number WO 2014/022411, U.S. Pat. Nos. 1,289,580, and 3,551,914.

Thus, there is a need for a passive ankle mechanism that is able to provide swing phase dorsiflexion as well as stance phase support (i.e. resist ground reaction forces during stance without yielding in the dorsiflexion direction) in a simple, durable, and compact form factor. Such a mechanism may prove useful in the application areas of ankle prostheses and orthoses. One application of this mechanism in the field of ankle orthotics is use in an orthosis for treating drop-foot gait in which swing phase ankle dorsiflexion is needed.

### SUMMARY

Various implementations include a leg prosthesis or orthosis device. The device includes a shank member, a foot member, a connecting link, and a sliding link. The connecting link is movably coupled to the shank member at a first location and is moveably coupled to the foot member at a second location. The sliding link is constrained to move along a sliding axis relative to the shank member. The sliding link is rotatably coupled to the foot member at a third location.

In some implementations, the third location is substantially in line with the sliding axis.

In some implementations, the movable coupling between the shank member and the connecting link has a single degree of freedom, and the movable coupling between the foot member and the connecting link has a single degree of freedom. In some implementations, the connecting link is rotatably coupled to the shank member at the first location, and the connecting link is rotatably coupled to the foot member at the second location. In some implementations, the connecting link is prismatically coupled to the shank member at the first location, and the connecting link is rotatably coupled to the foot member at the second location. In some implementations, the connecting link is rotatably coupled to the shank member at the first location, and the connecting link is prismatically coupled to the foot member at the second location.

In some implementations, the device further includes a compliant member for exerting a force on the foot member to urge the foot member toward a dorsiflexed position relative to the shank member. In some implementations, the compliant member is disposed between the sliding link and the shank member. In some implementations, the compliant member is disposed between the connecting link and one of the foot member or the shank member.

In some implementations, the device further includes a mechanical hard stop configured to limit the angular range of the rotatable coupling of the foot member to the sliding link in a plantarflexion direction. In some implementations, a direction of force that is transmitted through the connecting link from the foot member to the shank member is substantially perpendicular to the sliding axis of the sliding link when the mechanical hard stop is preventing further angular rotation of the rotatable coupling of the foot member to the sliding link in the plantarflexion direction. In some implementations, the connecting link is rotatably coupled to the shank member at the first location, and the connecting link is rotatably coupled to the foot member at the second location. In some implementations, an axis intersecting the first location and the second location is substantially perpendicular to the sliding axis when the mechanical hard stop is preventing further angular rotation of the rotatable coupling of the foot member to the sliding link in the plantarflexion direction.

In some implementations, the connecting link includes an eccentric shaft. In some implementations, a bearing surface of one of the first location or the second location encompasses an axis of rotation of an other of the second location or the first location as viewed in a plane perpendicular to the axis of rotation.

In some implementations, a sliding interface between the sliding link and the shank member is circular as viewed in a plane perpendicular to the sliding axis. In some implementations, a sliding interface between the sliding link and the shank member includes a sliding dovetail. In some implementations, a sliding interface between the sliding link and the shank member includes a first one-way fluid valve and a second one-way fluid valve. In some implementations, the first one-way fluid valve allows fluid flow into, and restricts fluid flow from, a space defined between the sliding link and the shank member. In some implementations, the second one-way fluid valve restricts fluid flow into, and allows fluid flow from, the space defined between the sliding link and the shank member.

### BRIEF DESCRIPTION OF DRAWINGS

Example features and implementations are disclosed in the accompanying drawings. However, the present disclosure is not limited to the precise arrangements and instrumentalities shown.

FIGS. 1A-1D are schematic views of devices including a shank member, a foot member, a sliding link, and a connecting link. The connecting link is movably coupled to both the foot member and the shank member through either a rotational joint or a sliding joint. The sliding link is further constrained to move along a sliding axis relative to the shank member, and the sliding link is coupled to the foot member via a rotational joint.

FIGS. 1E-1F are schematic illustrations in which the roles of the foot member and shank member are inverted. Furthermore, the connecting link is shown in such a way as to indicate that it may be coupled to the foot and shank members via rotational or sliding joints.

FIG. 2 is sagittal plane view of an implementation in which the shank member and foot member are visible.

FIG. 3 is a perspective view of the implementation shown in FIG. 2.

FIG. 4A is a perspective view of the implementation shown in FIG. 2, and FIG. 4B is a sagittal plane cutaway view of the implementation shown in FIG. 2. FIG. 4B also shows a compliant member that biases the foot member towards dorsiflexion relative to the shank member as well as a mechanical hard stop limiting the plantarflexion motion of the foot member relative to the shank member.

FIG. 5A is a perspective view of a device according to another implementation. FIG. 5B is a sagittal plane cutaway view of the device shown in FIG. 5A. The connecting link has been formed as an eccentric shaft, such that the bearing surface of either the first or second location of the connecting link encompasses the axis of rotation of the second or first location in the plane normal to the rotation axes.

FIG. 6 is a kinematic diagram of an implementation in which the connecting link is coupled to both the foot member and shank member via rotational joints. Note that in this kinematic diagram, the shank member is fixed to the ground. This diagram further includes mechanical hard stops that limit the rotational motion of the foot member relative to the shank member.

FIG. 7 is a side view of an implementation shown throughout various stages of the walking gait cycle. The approximate position of the ground reaction force is shown with the vertical arrow. At heel strike, the ankle mechanism is in a dorsiflexed configuration, and the posteriorly located ground reaction tends to move the ankle in the plantarflexion direction. During stance phase, the ground reaction force moves anteriorly, but the ankle mechanism does not yield in the dorsiflexion direction. During late stance, an elastic foot member in series with the device may deflect to store and release energy. During swing phase, when the ground reaction force is removed, the ankle returns to a dorsiflexed configuration to provide swing phase foot clearance.

FIGS. 8A and 8B are kinematic diagrams of an implementation in which the connecting link is movably coupled to both the foot member and the shank member through rotational joints. FIG. 8A shows the mechanism in a singular configuration. FIG. 8B shows the mechanism past the singular configuration relative to the mechanism configuration shown in FIG. 6.

FIG. 9A is a perspective view of the implementation of FIG. 7. FIG. 9B is a sagittal plane cross-sectional view of the implementation shown in FIG. 7. FIG. 9B also shows a compliant member that biases the foot member towards dorsiflexion relative to the shank member as well as a mechanical hard stop limiting the plantarflexion motion of the foot member relative to the shank member.

FIG. 10A is a perspective view of a device according to another implementation. FIG. 10B shows a sagittal plane cutaway view of the device of 10A. The device includes a connecting link that is movably coupled to the shank member with a sliding joint at a first location and movably coupled to the foot member with a rotational joint at a second location.

FIG. 11A is a perspective view of a device according to another implementation. FIG. 11B is a sagittal plane cutaway view of the device of 11A. The device includes a connecting link that is movably coupled to the shank member through a rotational joint at a first location and the connecting link is movably coupled to the foot member through a sliding joint at a second location.

FIGS. 12A-12F are implementations of the mechanism depicted in FIG. 1A in which a compliant member is shown connecting various components of the device.

FIG. 13 is an implementation of the mechanism depicted in FIG. 1A in which the sliding joint between the shank member and sliding link is configured as a fluid pump. The implementation shows two one-way fluid valves connected to the prismatic coupling between the shank member and sliding link. Furthermore, a fluid line is shown which may be connected to a fluid chamber in order to draw a vacuum in said fluid chamber upon cyclic motion of the mechanism.

### DETAILED DESCRIPTION

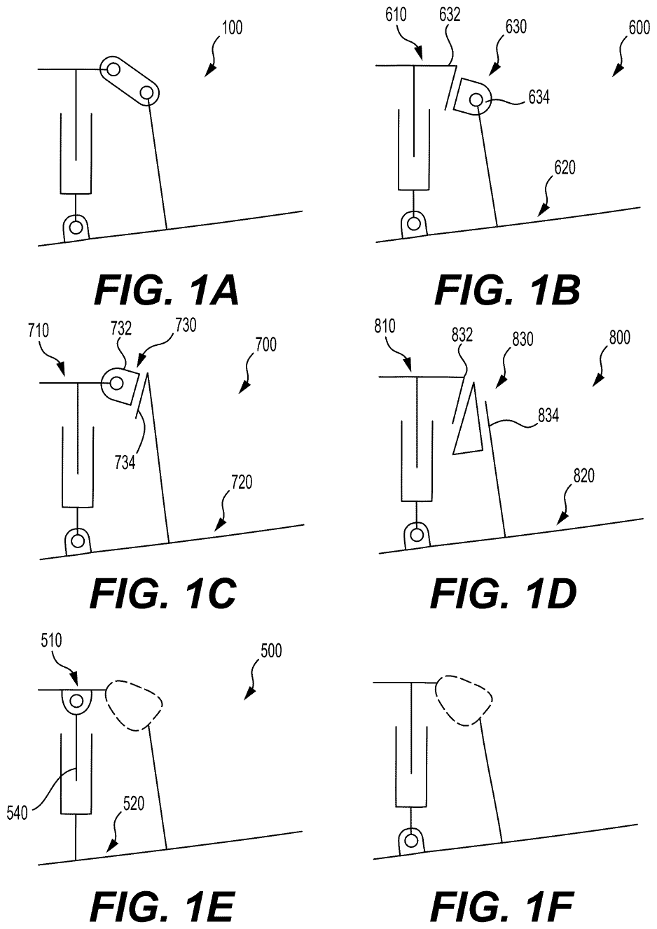

The devices, systems, and methods disclosed herein provide for prostheses and orthoses that include a shank member, a foot member, a connecting link, and a sliding link. The connecting link is movably coupled to the foot member at a first location and is movably coupled to the shank member at a second location. Furthermore, the sliding link is constrained to move along a sliding axis relative to the shank link, and the foot member is rotatably coupled to the sliding link. The movable coupling between the connecting link and either the foot or shank member may include either a rotational or prismatic joint. This mechanism may be inverted by switching the role of the shank member and foot member without substantially changing the function of the device, as shown in FIGS. 1E and 1F.

Various implementations include a leg prosthesis or orthosis device. The device includes a shank member, a foot member, a connecting link, and a sliding link. The connecting link is movably coupled to the shank member at a first location and is moveably coupled to the foot member at a second location. The sliding link is constrained to move along a sliding axis relative to the shank member and is rotatably coupled to the foot member at a third location.

In some implementations, the connecting link may be movably coupled to the shank member at a first location through either a rotational joint or a sliding joint. Furthermore, the connecting link may be movably coupled with the foot member through either a rotational joint or a sliding joint. Various combinations of these means of coupling the shank member, foot member and connecting link are shown schematically in FIGS. 1A-1D. FIG. 1A shows the connecting link coupled to both the foot and shank members through rotational joints. FIG. 1B shows the connecting link coupled to the shank member through a sliding joint and the foot member through a rotational joint. FIG. 1C shows the connecting link coupled to the shank member through a rotational joint and the foot member through a sliding joint. FIG. 1D shows the shank member coupled to both the foot and shank members through sliding joints.

In some implementations, a compliant member may be introduced in the mechanism, such that, when no external loads are placed on the device, the ankle adopts a dorsiflexed configuration. This compliant member may consist of a spring, elastomer, resilient material, compressed fluid, or other mechanism of providing a spring-like behavior. This compliant member may be linear or rotatory in nature and may be placed between various components of the mechanism. For example, a linear compliant member may be placed between the sliding link and the shank member to provide a spring-like force between these two elements.

In some implementations, a mechanical hard stop may be introduced in the mechanism, such that, the hard stop limits the angular range of motion of the foot member relative to the sliding link. The mechanical hard stop may limit the angular range of motion of the mechanism in at least one of the plantarflexion or dorsiflexion direction. The mechanical hard stop may be situated between various components of the mechanism. The mechanical hard stop may be situated between any two components of the mechanism. For example, the mechanical hard stop may be situated between the sliding link and the shank member, between the sliding link and the foot member, between the foot member and the connecting link, or between the connecting link and the shank member. It should be noted that this list of potential locations for the mechanical hard stop is not exhaustive.

In some implementations, a sliding joint within the mechanism may be configured as a fluid pump. A sliding joint within the mechanism may be configured as a fluid pump that may provide a vacuum pressure to a fluid chamber. The fluid chamber may be the space within a prosthetic socket or suspension system. In this case, the motion of the mechanism generates a vacuum pressure within the prosthetic socket or suspension system so as to assist with attaching the socket to the residual limb of the user. The fluid pump may be integrated into any prismatic joint within the mechanism such as the interface between the sliding link and the shank member. Alternatively, the fluid pump may be integrated into a sliding joint attached to the connecting link. The prismatic motion of the fluid pump may expel fluid (such as air) through a one-way valve and take in fluid through a separate one-way valve. In this way, a cyclic motion of the prismatic joint to which the fluid pump is integrated generates a fluid flow (and induced pressure differential) from the inlet valve to the outlet valve.

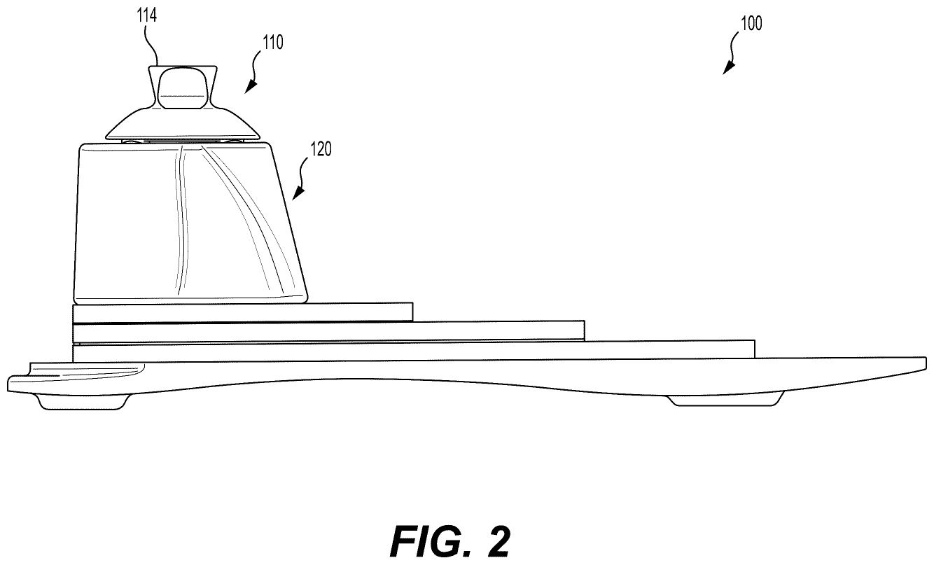

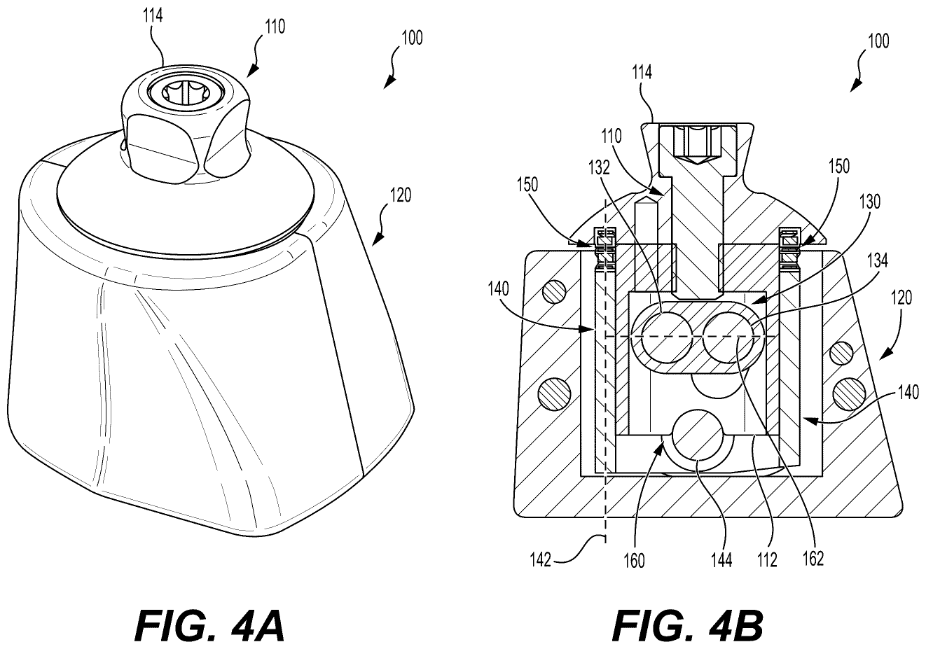

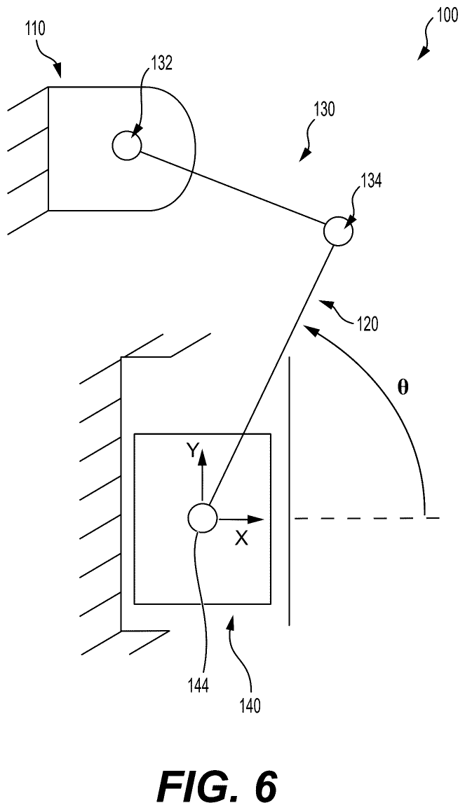

FIG. 2 shows an implementation of a leg prosthesis or orthosis device 100, and FIG. 4B shows a sagittal plane cutaway view of the device 100 shown in FIG. 2. The device 100 of FIG. 2 is depicted in schematic form in FIG. 6. The device 100 includes a shank member 110, a foot member 120, a connecting link 130, and a sliding link 140.

The shank member 110 includes a distal portion 112 and a proximal portion 114 configured to be coupled to the user.

The connecting link 130 is rotatably coupled to the shank member 110 at a first location 132. The rotatable coupling between the shank member 110 and the connecting link 130 has a single degree of freedom.

The connecting link 130 is also rotatably coupled to the foot member 120 at a second location 134. The rotatable coupling between the foot member 120 and the connecting link 130 also has a single degree of freedom.

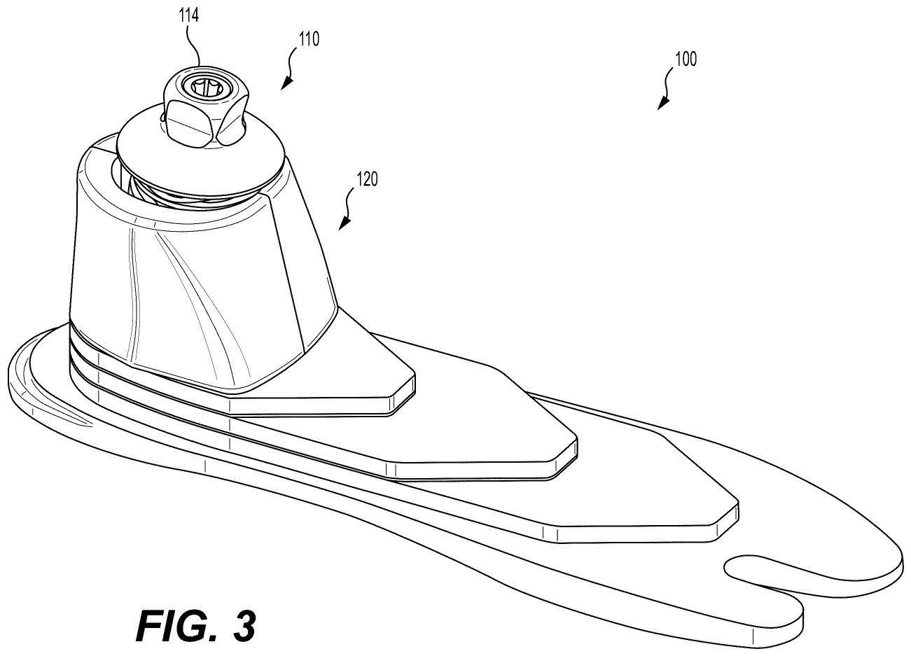

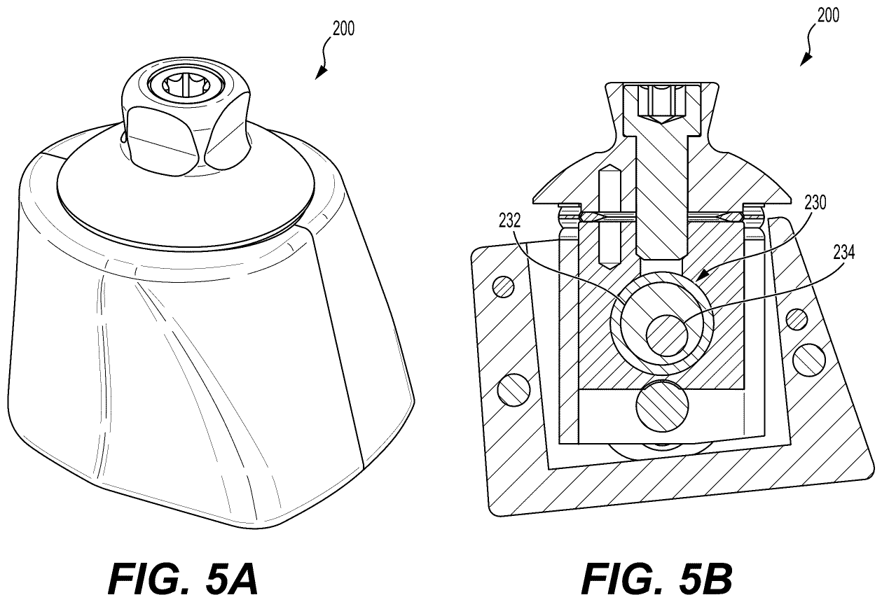

Although the connecting link 130 shown in FIGS. 2-4B is a linking member, in other implementations, such as the device 200 shown in FIGS. 5A and 5B, the connecting link 230 is formed as an eccentric shaft. The bearing surface of either the first location 232 or second location 234 encompasses the axis of rotation of the second location 234 or first location 232 in the plane normal to the rotation axes. This allows the eccentric shaft 230 to function as a linking member but does not constrain how close the first location 232 and second locations 234 are relative to each other. For example, in the implementation shown in FIGS. 5A and 5B, the axis of rotation of the second location 234 is encompassed by the shaft of the first location 232.

The sliding link 140 of the device 100 shown in FIGS. 2-4B is coupled to the shank member 110 by a prismatic joint such that the sliding joint is constrained to move along a sliding axis 142 relative to the shank member 110. The translational coupling between the shank member 110 and the sliding link 140 includes a shaft sliding in a cylindrical bearing.

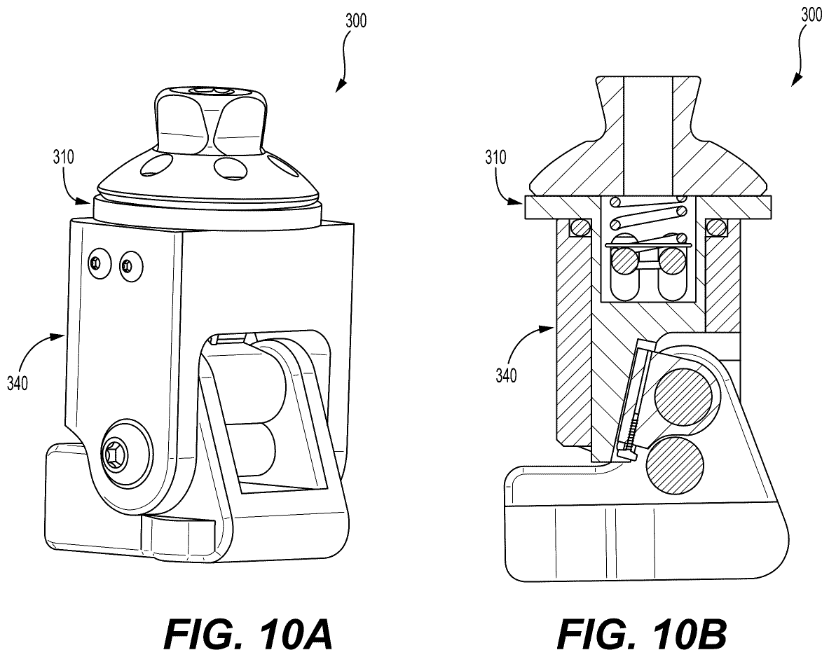

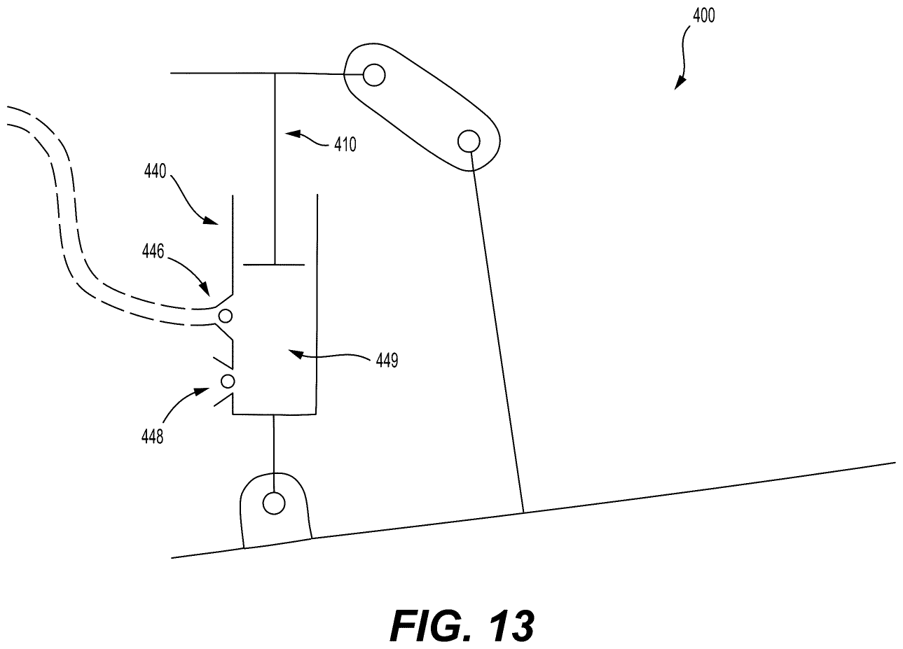

The sliding interface between the sliding link 140 and the shank member 110 is circular as viewed in a plane perpendicular to the sliding axis 142. However, in some implementations, such as the device 300 shown in FIGS. 10A and 10B, a sliding interface between the sliding link 340 and the shank member 310 includes a sliding dovetail. In some implementations, such as the device 400 shown in FIG. 13, a sliding interface between the sliding link 440 and the shank member 410 includes a first one-way fluid valve 446 and a second one-way fluid valve 448. The first one-way fluid valve 446 allows fluid flow into, and restricts fluid flow from, a space 449 defined between the sliding link 440 and the shank member 410. The second one-way fluid valve 448 restricts fluid flow into, and allows fluid flow from, the space 449 defined between the sliding link 440 and the shank member 410.

The sliding link 140 is rotatably coupled to the foot member 120 at a third location 144. The third location 144 is substantially in line with the sliding axis 142. However, in some implementations, such as the device 500 shown in FIG. 1E, the sliding link 540 is flipped such that the sliding link 540 is rotatably coupled to the shank member 510, and the sliding link 540 is prismatically coupled to the foot member 520.

It may be noted that the implementation shown in FIGS. 2-4B includes a slider-crank mechanism. However, in some implementations, such as the device 600 shown in FIG. 1B, the connecting link 630 is prismatically coupled to the shank member 610 at the first location 632, and the connecting link 630 is rotatably coupled to the foot member 620 at the second location 634. In some implementations, such as the device 700 shown in FIG. 1C, the connecting link 730 is rotatably coupled to the shank member 710 at the first location 732, and the connecting link 730 is prismatically coupled to the foot member 720 at the second location 734. In some implementations, such as the device 800 shown in FIG. 1D, the connecting link 830 is prismatically coupled to the shank member 810 at the first location 832, and the connecting link 830 is prismatically coupled to the foot member 820 at the second location 834.

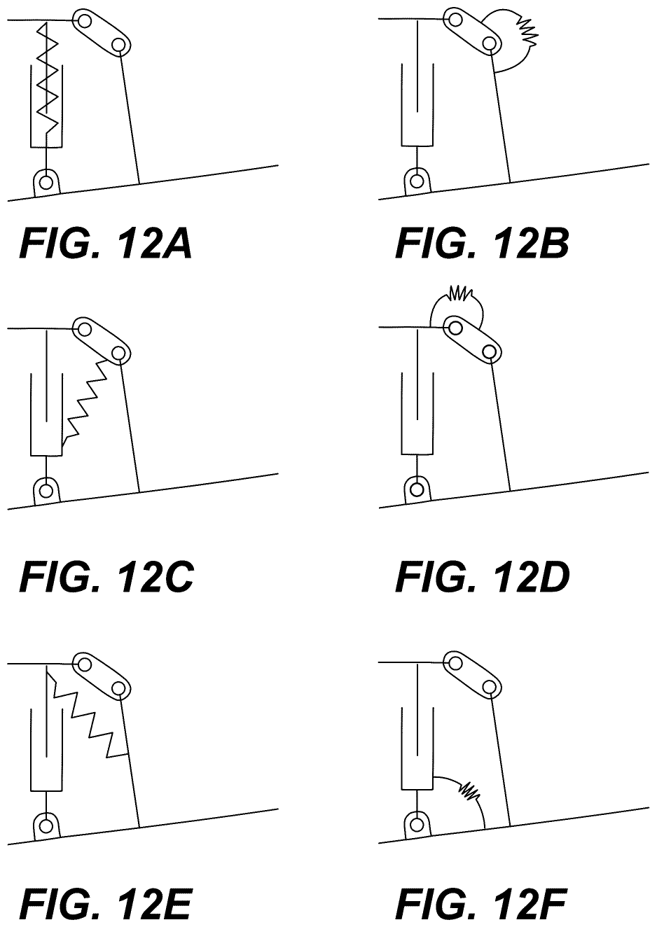

A compliant member 150, such as the spring shown in FIGS. 2-4B, is introduced between the shank member 110 and sliding link 140 to bias the mechanism toward a dorsiflexed configuration. However, in some implementations, the compliant member is disposed between the connecting link and one of the foot member or the shank member. FIGS. 12A-12F show configurations of a compliant member 150 included in various implementations of a device. FIG. 12A shows a compliant member between the shank member and the sliding member. FIG. 12B shows a compliant member between the connecting link and the foot member. FIG. 12C shows a compliant member between the sliding member and the connecting link. FIG. 12D shows a compliant member between the shank member and the connecting link. FIG. 12E shows a compliant member between the shank member and the foot member. FIG. 12F shows a compliant member between the sliding member and the foot member.

The device 100 further includes a mechanical hard stop 160 configured to limit the angular range of the rotatable coupling at the third location 144 of the foot member 120 to the sliding link 140 in a plantarflexion direction. The mechanical hard stop 160 is configured such that, when the mechanical hard stop 160 is preventing further angular rotation of the rotatable coupling of the foot member 120 to the sliding link 140 in the plantarflexion direction, an axis 162 intersecting the first location 132 and the second location 134 is substantially perpendicular to the sliding axis 142. In this position, a direction of force that is transmitted through the connecting link 130 from the foot member 120 to the shank member 110 is substantially perpendicular to the sliding axis 142 of the sliding link 140.

The configuration of components in this mechanism creates a single degree of freedom mechanism in which a translational motion of the shank member relative to the sliding link is kinematically coupled to a rotational motion of the foot member relative to the sliding link. As such, in this mechanism, the foot member both rotates and translates relative to the shank member. Specifically, relative to the shank, the foot member undergoes a planar rotation and a prismatic translation along a single axis. The resulting motion of the foot, therefore, only spans two dimensions (i.e., translation in a single axis and pure rotation) in a reference frame attached to the shank. Through this kinematic coupling, external forces applied to the shank member along the axis of the prismatic degree of freedom between the shank member and the sliding link are transduced to torques about the rotational coupling between the foot member and the sliding link. In other words, forces along the translational axis of the shank member are transduced into torques about the rotational axis of the foot member on the sliding link. When a force is applied to the shank member along the translational axis of its coupling with the sliding link, the resulting torque that is generated about the rotational axis of the foot member (on the sliding link) is governed by the mechanical advantage of the mechanism in its current configuration. This mechanical advantage can be described using the principle of virtual work where a compressive force, F, may be applied to the translational joint between the sliding link and the shank member across an infinitesimal linear translation, dy. This applied compressive force results in a torque, −r, about the rotational coupling between the sliding link and foot member being applied across an infinitesimal rotational displacement, −dθ. The expression of energy flow through the mechanical system can be expressed as a statement of virtual work (1).

$$F dy = vd θ \qquad ( 1 )$$

This expression of virtual work, (1), may then be rearranged provide a description of the mapping between torques and forces on the mechanism (2).

$$τ = F \frac{dy}{d θ} \qquad ( 2 )$$

As can be seen from (2), translational forces applied between the sliding link and the shank member can be mapped to torques about the rotational coupling between the foot member and the sliding link by multiplying by the derivative,

$$\frac{dy}{d θ} .$$

As such, this derivative,

$$\frac{dy}{d θ} ,$$

describes the mechanical advantage of the mechanism. It should be noted that the mechanical advantage of the mechanism may change based on the configuration of the mechanism. In other words, the derivative,

$$\frac{dy}{d θ} ,$$

may be (and generally is) a function of the mechanism configuration. The proportions of the mechanism described herein may be chosen such that the mechanical advantage of the mechanism exhibits beneficial behaviors during walking.

Recall that the goals of the implementations disclosed herein are to passively dorsiflex the ankle during the swing phase of gait, plantarflex to a nominal position during the heel strike phase of gait and provide supportive torques to the user during the stance phase of gait (i.e., resist applied ground reaction forces without yielding in the dorsiflexion direction).

Considering the swing phase of gait, a dorsiflexed configuration of the ankle is achieved through the compliant member, which, when the ankle is unloaded, tends to force the ankle into a dorsiflexed position. At a heel strike event, the ground reaction force is posterior to the rotational coupling between the foot member and sliding link such that a plantarflexive torque about this joint is generated by the ground reaction force. Additionally, the component of the ground reaction force that is in-line with the translational axis of the shank member relative to the foot member will tend to compress this joint. The compression of the shank member relative to the sliding link will tend to plantarflex the foot member relative to the sliding link due to the connecting link. As such, at a heel strike event, the ankle will tend to undergo a plantarflexion motion until it reaches a mechanical hard stop.

When the ankle plantarflexes at heel strike, the configuration of the mechanism changes, and so does the mechanical advantage of the mechanism (which can be quantitively measured by the derivative,

$$\frac{dy}{d θ}$$

During the stance phase of gait, the effective point of application of the ground reaction force (i.e. the center of pressure) progresses anteriorly towards the toe, and as such, begins to create a torque in the dorsiflexion direction on the foot member about the rotational joint between the foot member and the sliding link at later stages of stance. At the same time, however, the compressive force on the shank member due to the ground reaction force induces a plantarflexion torque on the foot member relative to the sliding link through the mechanical advantage of the mechanism. The direction of the net torque on the foot member relative to the sliding link may be determined by summing the moments about the axis of the rotational coupling between the foot member and the sliding link as expressed in (3).

$$\sum {N}_{A} = - F \frac{dy}{d θ} + τ \qquad ( 3 )$$

If it is assumed that the ground reaction force during stance is located at a distance, from the axis of rotation of the foot member relative to the sliding link (row is the anterior distance of the center of pressure in front of the rotational joint coupling the sliding link and foot member), that the force provided by the compliant member is small relative to the ground reaction force, and that the ground reaction force is coaxial with the translational axis between the shank member and the sliding link, then the torque induced by the ground reaction force is approximately equal to r_{ccp}F. Substituting this expression (r=r_{ccp})) into (3) yields (4).

$$\sum {N}_{A} = - F \frac{dy}{d θ} + {τ n}_{\text{?}} F \qquad ( 4 )$$

$$\text{?} \text{indicates text missing or illegible when filed}$$

In order for the mechanism to not yield in the dorsiflexion direction during the stance phase of gait, the summation expressed in (4) must be less than or equal to zero (ΣM_{d}≤0). Stated alternatively, the plantarflexive torque generated by the compressive load placed on the mechanism must be larger in magnitude than any dorsiflexive torque generated by a distal location of the center of pressure. Imposing this inequality constraint on (4) and simplifying the expression yields (5).

$$\frac{dy}{d θ} \text{?} {r}_{\text{?}} \qquad ( 5 )$$

$$\text{?} \text{indicates text missing or illegible when filed}$$

If the forefoot is assumed to be of length, l_{feet}, may be noted that r_{ccp} cannot feasibly be longer than l_{feet} (the center of pressure cannot extend past the end of the foot). As such, the stability criterion expressed in (5) may be satisfied if, in the plantarflexed configuration,

$$\frac{dy}{d θ} \geq {l}_{foot} .$$

If this stability criterion is satisfied, then the ankle mechanism will not yield in the dorsiflexion direction during the stance phase of walking, and the user will experience a supportive torque. If the ankle mechanism is placed in series with an elastic foot element, during the stance phase of gait, the elastic foot element can store and subsequently release energy at terminal stance. At terminal stance, when the ankle mechanism is unloaded, the compliant member (within the ankle mechanism) will bias the ankle towards dorsiflexion, lifting the toe to provide foot clearance during the swing phase of gait.

The configuration of components within the devices disclosed herein may be designed to achieve the stability criterion. Namely, the rotation of the foot member relative to the sliding link is coupled to the prismatic motion of the shank member relative to the sliding link due to the connecting link. As such, forces on the shank member along the translational axis of the sliding link induce torques about the rotational coupling between the foot member and the sliding link. The dimensions of these components may then be selected such that the mathematical stability criterion is achieved. Alternatively, the dimensions of the various components may be selected so that other desired behavior is achieved via the kinematic coupling between the translation and rotation of the foot member relative to the shank member.

The stability criterion described above may be achieved by appropriately selecting the dimensions of the various components. In some implementations, when the device is against a mechanical hard stop limiting the plantarflexion rotation of the foot member relative to the sliding link, the force transmitted through the connecting link (neglecting any friction forces at the interfaces between the connecting link and either the shank or foot members) is substantially perpendicular to the prismatic axis of the sliding link. When the force through the connecting link (neglecting frictional forces) is substantially perpendicular to the prismatic axis of the sliding link, the mechanical advantage of the mechanism is very large, and the stability criterion is met. In a specific embodiment in which the connecting link is rotatably coupled to the shank member at a first location and rotatably coupled to the foot member at a second location, the stability criterion can be achieved through proper arrangement of components. In these implementations, the stability criterion can be achieved if, when the device is against a mechanical hard stop limiting the plantarflexion rotation of the foot member relative to the sliding link, a line that intersects the centers of the rotational couplings between the connecting link and the shank and foot members is substantially perpendicular to the prismatic axis of the sliding link.

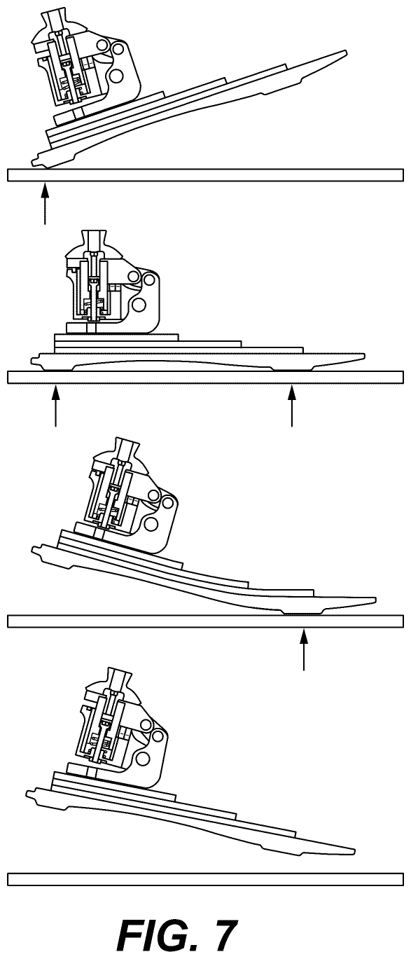

To summarize the function of this device, during the swing phase of gait, a compliant member biases the ankle mechanism towards dorsiflexion. During heel strike, the ground reaction force tends to plantarflex the ankle mechanism. During stance phase, the ankle remains locked and does not yield in the dorsiflexion direction due to the configuration of the mechanism. Finally, when the ankle is unloaded, the compliant member dorsiflexes the ankle to provide toe clearance. A model of a preferred embodiment may be seen in these various phases of gait in FIG. 7.

The prismatic joint between the sliding link and the shank member provides a number of benefits to the device that enhance its performance as well as practical usefulness. In the devices disclosed herein, the foot member both rotates and translates relative to the shank member. By utilizing a prismatic joint to movably couple the sliding link and the shank member and a rotational joint to movably couple the sliding link and the foot member, the rotation and translation of the foot member relative to the shank member can be treated independently with respect to sealing the mechanism from dirt, debris, or water. Specifically, the prismatic linear joint can be sealed from the elements independently from the sealing solution used for the rotational coupling between the foot member and sliding link. Therefore, this arrangement of components allows for relatively simple sealing or waterproofing of components. The prismatic coupling between the shank member and sliding link also allows for a standard linear spring to be used as the compliant member. Linear springs are widely available, allowing for the properties of this compliant member to be selected from a large selection of commercially available parts. As such, the properties of the compliant element may be easily catered or modified for different users of the device. For example, a heavier user may require a stiffer compliant element. The prismatic joint between the sliding link and the shank member allows for standard springs to be swapped easily to achieve different levels of compliance. Furthermore, the use of a prismatic joint to couple the sliding link and the shank member allows for the introduction of fluid pumping functionality as previously described. The prismatic motion of the sliding link relative to the shank member is similar to the prismatic motion required of many standard fluid pumps (or vacuum pumps). Thus, this translational motion can be harnessed to create a fluid pump via the introduction of two separate one-way valves.

Moreover, if the prismatic coupling between the sliding link and the shank member is coaxial with the axis of the shank, then during the motion of the mechanism, the foot member will only translate relative to the shank along an axis that is parallel to the shank. In other words, the foot will not move horizontally relative to the shank. Any horizontal translation of the foot member relative the shank member during the motion of the device may serve to impair the stability of the user. Namely, a horizontal translation of the foot member relative to the shank member during contact with the ground may serve to destabilize the user or even lead to a fall. The prismatic coupling between the shank member and the sliding link ensures that the foot will not experience any horizontal translation relative to the shank member during motion of the device.

Another beneficial property of the prismatic coupling between the shank member and the sliding link is that, if the prismatic axis of the sliding link is parallel to the axis of the shank, then the stability criterion described previously will be reached after ground contact while minimizing the amount of required vertical motion of the foot member relative to the shank member. Recall that the goal of the device is to prevent falls during gait by promoting clearance between the foot and the ground during swing phase. During swing phase, the foot member rotates in a dorsiflexion direction relative to the shank member, thereby lifting the toes to provide ground clearance. Simultaneously, however, the foot member translates relative to the shank member in such a way as to make the leg longer. To provide the most ground clearance, the mechanisms of the devices disclosed herein are designed to achieve a significant amount of dorsiflexion rotation and a minimal amount of leg-lengthening translation. A prismatic joint between the shank member and the sliding link (in which the prismatic axis of the slider is parallel to the shank) maximizes the sensitivity of the mechanical advantage to changes in leg length. Stated alternatively, by utilizing a prismatic coupling between the shank member and the sliding link, the stance stability criterion can be achieved with a minimum amount of axial translation of the foot member relative to the shank member, thereby optimizing the tradeoff between rotation and translation of the foot member.

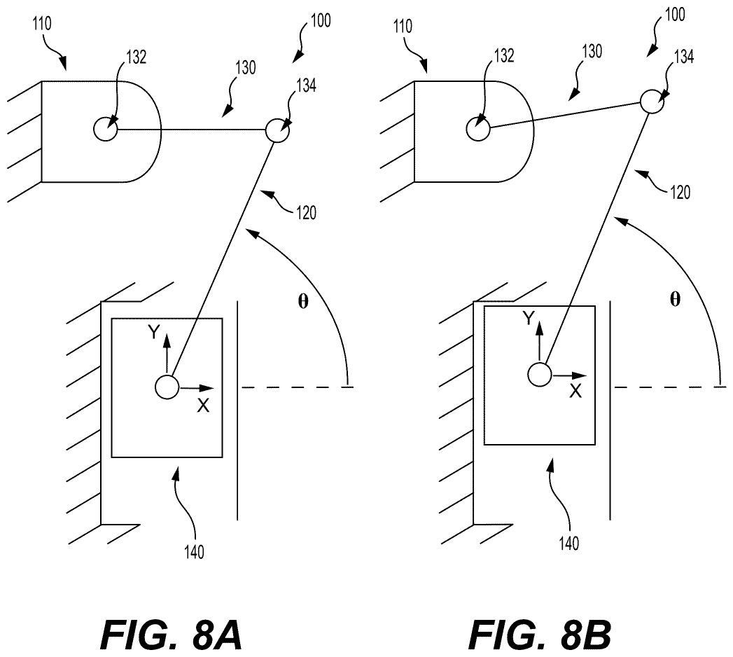

It may be noted that, as the mechanism moves from a fully dorsiflexed position towards plantarflexion, the mechanism approaches a singular configuration. In the case of the preferred embodiment, in which the ankle is a slider crank mechanism (the connecting link is movably coupled to both the shank and foot members via rotational joints), the singular configuration occurs when the line intersecting the two rotational axes of the connecting link is perpendicular to the translational axis of the sliding link (as shown in FIG. 8A). As the mechanism approaches this singular configuration, the mechanism's mechanical advantage,

$$\frac{dy}{d θ} ,$$

approaches infinity. Therefore, if the mechanism is sufficiently close to the singular position when it contacts the plantarflexion hard stop, the ankle mechanism achieves the locking criteria for any sized foot (as the center of pressure can never be located at an infinite distance from the ankle joint).

If the ankle mechanism moves through the singular configuration, additional compression of the shank member relative to the sliding link will cause the foot member to dorsiflex relative to the sliding link rather than plantarflex. One consequence of passing through the mechanism's singular configuration is that the sign of the mechanical advantage,

$$\frac{dy}{d θ} ,$$

changes. Once past the singular configuration, dorsiflexive torques about the rotational axis between the foot member and the sliding link will tend to compress the prismatic joint between the shank member and the sliding link. As such, if the mechanism passes through the singular configuration during the heel strike phase of gait, both compressive loads applied to the shank as well as distal ground reaction forces applied to the foot member will tend to compress the prismatic joint between the shank member and sliding link. A mechanical hard stop may then be implemented in the prismatic joint between the shank member and sliding link such that ankle does not yield in the dorsiflexion direction during the stance phase of gait. An exemplary kinematic diagram illustrating an implementation passing through the singular configuration is depicted in FIG. 8B.

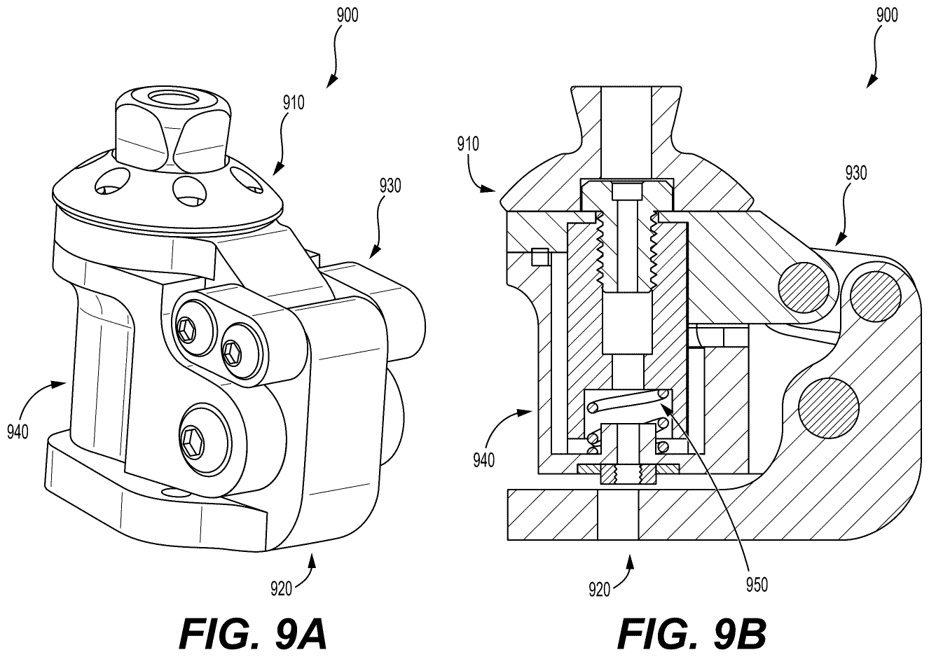

Various embodiments of the present disclosure are pictured in FIGS. 9-11. The presented examples are not an exhaustive list of possible implementations and should not be viewed as limiting. FIG. 9 shows an implementation of a device 900 in a configuration in which the connecting link 930 is movably coupled to both the shank member 910 and the foot member 920 through a rotational coupling. Furthermore, a compliant member 950 (linear spring) is placed between the shank member 910 and the sliding link 940. FIG. 9 shows an isometric view of the mechanism 900 (FIG. 9A) as well as a sagittal plane cutaway view of the mechanism 900 (FIG. 9B).

FIG. 10 shows an implementation in which the connecting link is movably coupled to the shank member via a prismatic joint (specifically a sliding dovetail joint) and is movably coupled to the foot member via a rotational joint. A compliant member (linear spring) is placed between the shank member and the linear slider. FIG. 10 shows an isometric view of the mechanism (FIG. 10A) as well as a sagittal plane cutaway view of the mechanism (FIG. 10B).

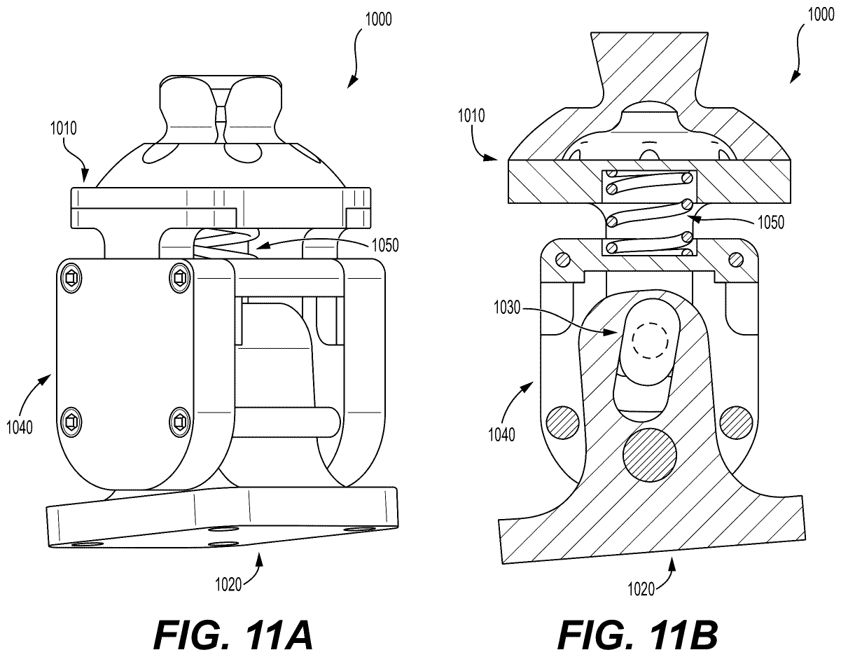

FIG. 11 shows an implementation of a device 1000 in which the connecting link 1030 is movably coupled to the shank member 1010 via a rotational joint and is movably coupled to the foot member 1020 via a prismatic joint. Furthermore, the prismatic joint coupling the shank member 1010 and sliding link 1040 is implemented as linear sliding rails. Additionally, a compliant member 1050 (linear spring) is placed between the shank member 1010 and the sliding link 1040. FIG. 11 shows an isometric view of the mechanism 1000 (FIG. 11A) as well as a sagittal plane cutaway view of the mechanism 1000 (FIG. 11B).

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A leg prosthesis or orthosis device, the device comprising:
a shank member;
a foot member;
a connecting link movably coupled to the shank member at a first location and moveably coupled to the foot member at a second location;
a sliding link constrained to move along a sliding axis relative to the shank member, wherein the sliding link is rotatably coupled to the foot member at a third location.

2. The device of claim 1, wherein the third location is substantially in line with the sliding axis.

3. The device of claim 1, wherein the movable coupling between the shank member and the connecting link has a single degree of freedom, and wherein the movable coupling between the foot member and the connecting link has a single degree of freedom.

4. The device of claim 1, wherein the connecting link is rotatably coupled to the shank member at the first location, and wherein the connecting link is rotatably coupled to the foot member at the second location.

5. The device of claim 1, wherein the connecting link is prismatically coupled to the shank member at the first location, and wherein the connecting link is rotatably coupled to the foot member at the second location.

6. The device of claim 1, wherein the connecting link is rotatably coupled to the shank member at the first location, and wherein the connecting link is prismatically coupled to the foot member at the second location.

7. The device of claim 1, further comprising a compliant member for exerting a force on the foot member to urge the foot member toward a dorsiflexed position relative to the shank member.

8. The device of claim 7, wherein the compliant member is disposed between the sliding link and the shank member.

9. The device of claim 7, wherein the compliant member is disposed between the connecting link and one of the foot member or the shank member.

10. The device of claim 1, further comprising a mechanical hard stop configured to limit the angular range of the rotatable coupling of the foot member to the sliding link in a plantarflexion direction.

11. The device of claim 10, wherein a direction of force that is transmitted through the connecting link from the foot member to the shank member is substantially perpendicular to the sliding axis of the sliding link when the mechanical hard stop is preventing further angular rotation of the rotatable coupling of the foot member to the sliding link in the plantarflexion direction.

12. The device of claim 10, wherein the connecting link is rotatably coupled to the shank member at the first location, and wherein the connecting link is rotatably coupled to the foot member at the second location, and wherein an axis intersecting the first location and the second location is substantially perpendicular to the sliding axis when the mechanical hard stop is preventing further angular rotation of the rotatable coupling of the foot member to the sliding link in the plantarflexion direction.

13. The device of claim 1, wherein the connecting link comprises an eccentric shaft, wherein a bearing surface of one of the first location or the second location encompasses an axis of rotation of an other of the second location or the first location as viewed in a plane perpendicular to the axis of rotation.

14. The device of claim 1, wherein a sliding interface between the sliding link and the shank member is circular as viewed in a plane perpendicular to the sliding axis.

15. The device of claim 1, wherein a sliding interface between the sliding link and the shank member comprises a sliding dovetail.

16. The device of claim 1, wherein a sliding interface between the sliding link and the shank member comprises a first one-way fluid valve and a second one-way fluid valve, wherein the first one-way fluid valve allows fluid flow into, and restricts fluid flow from, a space defined between the sliding link and the shank member, and wherein the second one-way fluid valve restricts fluid flow into, and allows fluid flow from, the space defined between the sliding link and the shank member.
